(19) United States
(12) Patent Application Publication
(10) Pub. No.: US 2015/0250650 A1
(43) Pub. Date: Sep. 10, 2015

(54) APPARATUS AND METHOD FOR PERFORMING SURGICAL TREATMENTS OF THE EYE

(71) Applicant: SCHWIND eye-tech-solutions GmbH & Co. KG, Aschaffenburg (DE)

(72) Inventors: Samuel Arba MOSQUERA, Aschaffenburg (DE); Mario SHRAIKI, Stockstadt (DE)

(21) Appl. No.: 14/642,048

(22) Filed: Mar. 9, 2015

(30) Foreign Application Priority Data
Mar. 10, 2014 (EP) 14158612.3

(51) Int. Cl. A61F 9/008

(52) U.S. Cl. CPC A61F 9/00814; A61F 9/0084; A61F 2009/00872; A61F 2009/00853; A61F 2009/0087; A61F 2009/00887; A61F 2009/00891; A61F 2009/00895; A61F 9/00836

(57) ABSTRACT

The present invention relates to an apparatus and a method for performing surgical treatments of the eye, in particular of the eye cornea, including a laser device, which is arranged to emit pulsed treatment radiation having a wavelength between about 380 nm and 425 nm and a pulse duration between 0.1 ps and 10 ns, wherein the pulse energy of the treatment radiation is between 0.1 nJ and 5 μJ.

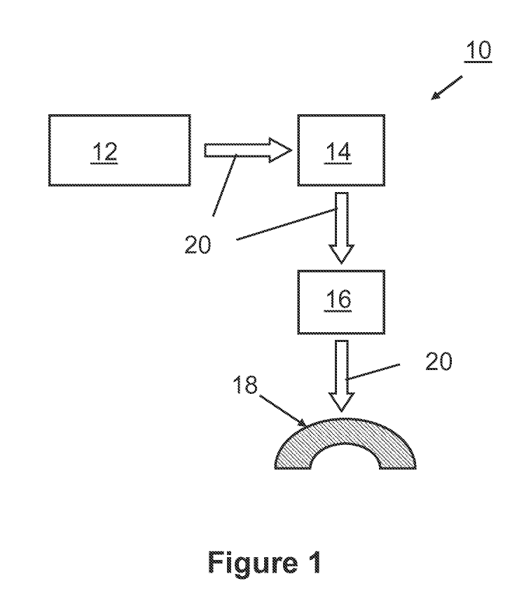

### FIELD OF THE INVENTION

The invention relates to an apparatus for performing surgical treatments of an eye, in particular the eye cornea, including a laser device, which is arranged to emit pulsed treatment radiation of a predetermined wavelength.

### BACKGROUND OF THE INVENTION

Such apparatuses are known. Thus, DE 101 48 783 A1 describes an assembly for the non-invasive optical processing of tissues of the eye, in particular for the refractive corneal surgery. The assembly includes a pulsed laser, which is suitable to emit radiation in a wavelength range between 500 nm and 1200 nm, wherein the pulse duration of the individual pulses is in the order of femtoseconds and the energy of the individual pulse is in the order of nanojoules. However, this assembly and the method for corneal surgery associated therewith are disadvantageous in that the transmission of various components of the anterior eye section considerably increases from a wavelength of ca. 425 nm. Thus, a predominant portion of the employed radiation or radiation energy can get to the eye lens and up to the retina. The risk of unintended damage of these eye components exists in the use of wavelength ranges between 500 nm and 1200 nm.

EP 1 787 607 B1 discloses a further assembly for performing surgical laser treatments of the eye cornea. Therein, the laser is to emit treatment radiation having a wavelength in the near UV range between 340 nm and 360 nm and a pulse duration in the femtosecond range, wherein the pulse energy of the treatment radiation is to be between 0.1 nJ and 5 μJ. However, this assembly is disadvantageous in that the absorption of the corneal epithelium is relatively severe in this proposed wavelength range. This means that correspondingly more radiation energy has to be expended to be able to deposit an amount of energy required for corneal ablation in the corresponding areas of the cornea. However, thereby, the risk of photokeratitis, i.e. a damage of the cornea by UV radiation or various forms of the epitheliopathy considerably increases.

From DE 10 2007 028 042 B3, an apparatus with a laser for processing a transparent material by non-linear absorption of pulsed laser radiation having a wavelength in a range from 300 to 1000 nm and a pulse length in a range from 300 ps to 20 ns effected in the area of the laser focus is known. By incorporating this mentioned wide wavelength range, the disadvantages described above either cannot be prevented with certainty in this known apparatus too, as far as it is applied within the scope of the treatment of the eye.

Therefore, it is the object of the present invention to provide an apparatus for performing surgical treatments of an eye, in particular of the eye cornea, of the initially mentioned kind, which is characterized by increased energy efficiency and improved safety with respect to unintended damages of components of the eye.

### SUMMARY OF THE INVENTION

According to the invention, this object is solved by an apparatus having the features of claim 1 as well as a method having the features of claim 11. Advantageous configurations with convenient developments of the invention are specified in the respective dependent claims.

An apparatus according to the invention for performing surgical treatments of an eye, in particular the eye cornea, includes a laser device, which is arranged to emit pulsed treatment radiation having a wavelength between about 380 nm and 425 nm and a pulse duration between 0.1 ps and 10 ns, wherein the pulse energy of the treatment radiation is between 0.1 nJ and 5 μJ.

The method according to the invention for surgical treatment of an eye, in particular the eye cornea, includes the following method steps:
a) providing a laser device, which is arranged to emit pulsed treatment radiation having a wavelength between about 380 nm and 425 nm and a pulse duration between 0.1 ps and 10 ns, wherein the pulse energy of the treatment radiation is between 0.1 nJ and 5 μJ; and
b) ablating predefined areas of the eye, in particular of the eye cornea, by means of the treatment radiation.

It has become apparent that using the mentioned laser parameters, the risk of unintended damages of components of the eye not to be treated is considerably reduced. In particular the use of wavelength ranges in the visible range ensures considerable reduction of the absorption in the corneal epithelium. Thereby, the energy expenditure decreases, which is required to deposit an energy demand required for corneal ablation in the areas of the cornea to be treated. The risk of photokeratitis is nearly excluded. Furthermore, by the wavelength range of 380 nm to 425 nm used according to the invention, a too severe transmission of various components of the anterior eye sections is excluded. A predominant portion of the employed radiation can therefore not get up to the eye lens or up to the retina, whereby the risk of unintended damage of these eye components is also nearly excluded. According to the invention, advantageously, the pulse duration can be predominantly adjusted in the picosecond range and the pulse energy of the treatment radiation can be adjusted between 0.1 nJ and 5 μJ. The advantages of the use of ultra-short laser pulses with pulse energies as low as possible are known.

In particular In particular, the wavelength of the treatment radiation can be between 380 and 405 nm. The transmission of the various components of the anterior eye section is again lower at an upper wavelength limit of 405 nm than at 425 nm, whereby advantages arise for certain surgical applications of the eye cornea or other components of the anterior eye section. Furthermore, it could be surprisingly determined that at a wavelength of the pulsed treatment radiation of approximately 380 nm, particularly good results arise in the glaucoma treatment or the so-called “cross-linking” of the cornea. Herein, riboflavin is for example used as an additive for the corneal collagen cross-linking with additional irradiation of the cornea. Therein, the pulse energy can be varied between 0.1 nJ and 5 μJ. The pulse duration can also be adjusted in the range according to the invention between 0.1 ps and 10 ns. Particular advantages additionally arise with pulse durations between 0.1 ps and 250 ps, since by the use of ultra-short laser pulses, lower portions of mechanical energy are introduced into the treated areas of the eye. The pulse duration range according to the invention of 0.1 ps to 250 ps means that the concerned pulse duration can be adjusted to the following values: 0.1 ps, 0.2 ps, 0.3 ps, 0.4 ps, 0.5 ps, 0.6 ps, 0.7 ps, 0.8 ps, 0.9 ps, 1.0 ps, 2 ps, 3 ps, 4 ps, 5 ps, 6 ps, 7 ps, 8 ps, 9 ps, 10 ps, 20 ps, 25 ps, 30 ps, 35 ps, 40 ps, 45 ps, 50 ps, 55 ps, 60 ps, 65 ps, 70 ps, 75 ps, 80 ps, 85 ps, 90 ps, 95 ps, 100 ps, 105 ps, 110 ps, 115 ps, 120 ps, 125 ps, 130 ps, 135 ps, 140 ps, 145 ps, 150 ps, 155 ps, 160 ps, 165 ps, 170 ps, 175 ps, 180 ps, 185 ps, 190 ps, 195 ps, 200 ps, 205 ps, 210 ps, 215 ps, 220 ps, 225 ps, 230 ps, 235 ps, 240 ps, 245 ps, 250 ps. Intermediate values are also conceivable. Furthermore, it has proven advantageous to use a wavelength of approximately 383 nm. By the variation of the pulse duration, photodisruptions can be achieved in different energy ranges. Thus, in the pulse duration range between 0.1 ps and 10 ps, and in particular in the pulse duration range between 0.6 ps and 10 ps, photodisruptions, in particular in the eye corneal area, can be achieved, which incorporate a very high energy density and generate a so-called “high density plasma”. So-called plasma radiance arises, which is equated with the threshold for the so-called optical breakdown, as is known. Photodisruptions by means of a plasma with very high energy density can be used for a deeper and wider ablation for example of the eye cornea. Areas of the cornea can be cut as so-called flaps or rings, in addition, there is the possibility of forming pockets in the cornea, which can for example be filled with artificial lenses. The apparatus according to the invention as well as the method according to the invention can also be used for the keratoplasty with the above mentioned parameters. Overall, besides the wavelength of the pulsed treatment radiation, which is at approximately 383 nm, it is also possible to use wavelengths in the entire inventive range between 380 and 425 nm. With the provision according to the invention of a treatment radiation of approximately 383 nm, a pulse energy range between 0.1 nJ and 5 μJ and with longer pulse durations in the range of 1 ps to 1 ns, in particular of 1 ps to 250 ps, photodisruption is effected in the transitional range between a relatively high energy density in the plasma and a low energy density in the arising plasma (“low density plasma”). The photodisruption is effected in the transitional range between a radiant and a non-radiant plasma. Due to the slightly lower energy density, material ablations can be more sharply defined. Herein too, flaps, rings or pockets can again be generated in the eye cornea. An apparatus according to the invention as well as the method according to the invention can be again used for the keratoplasty with the last mentioned laser parameters. The pulse duration range of 1 ps to 250 ps according to the invention means that the concerned pulse duration can be adjusted to the following values: 1.0 ps, 2 ps, 3 ps, 4 ps, 5 ps, 6 ps, 7 ps, 8 ps, 9 ps, 10 ps, 20 ps, 25 ps, 30 ps, 35 ps, 40 ps, 45 ps, 50 ps, 55 ps, 60 ps, 65 ps, 70 ps, 75 ps, 80 ps, 85 ps, 90 ps, 95 ps, 100 ps, 105 ps, 110 ps, 115 ps, 120 ps, 125 ps, 130 ps, 135 ps, 140 ps, 145 ps, 150 ps, 155 ps, 160 ps, 165 ps, 170 ps, 175 ps, 180 ps, 185 ps, 190 ps, 195 ps, 200 ps, 205 ps, 210 ps, 215 ps, 220 ps, 225 ps, 230 ps, 235 ps, 240 ps, 245 ps, 250 ps. Intermediate values are also conceivable. Again, besides a wavelength of the treatment radiation of approximately 383 nm, the entire wavelength range according to the invention from 380 to 425 nm can also be used. Finally, with pulsed treatment radiation having a wavelength of about 383 nm, a pulse energy of the treatment radiation between 0.1 nJ and 5 μJ and pulse durations between 0.1 ns and 10 ns, in particular 0.1 ns to 0.25 ns, a photodisruption of the corneal areas to be treated can be effected with a plasma of low energy density, that is a so-called non-radiant plasma. Herein, the areas of the photodisruption can be particularly exactly defined. Again, with an apparatus according to the invention as well as the method according to the invention with the last mentioned laser parameters, flaps, rings and pockets can be generated in the eye cornea. The apparatus as well as the method according to the invention can also be used for the keratoplasty. The pulse duration range according to the invention of 0.1 ns to 10 ns means that the concerned pulse duration can be adjusted to the following values: 0.10 ns, 0.15 ns, 0.20 ns, 0.25 ns, 0.30 ns, 0.35 ns, 0.40 ns, 0.45 ns, 0.50 ns, 0.55 ns, 0.60 ns, 0.65 ns, 0.70 ns, 0.75 ns, 0.80 ns, 0.85 ns, 0.90 ns, 0.95 ns, 1.0 ns, 2.0 ns, 2.5 ns, 3.0 ns, 3.5 ns, 4.0 ns, 4.5 ns, 5.0 ns, 5.5 ns, 6.0 ns, 6.5 ns, 7.0 ns, 7.5 ns, 8.0 ns, 8.5 ns, 9.0 ns, 9.5 ns, 10.0 ns. Intermediate values are also conceivable. Of course, besides the wavelength of the treatment radiation of 383 nm, with the mentioned ranges of the pulse energy of the treatment radiation between 0.1 nJ and 5 μJ and the pulse duration range between 0.1 ns and 10 ns, in particular 0.1 ns and 0.25 ns, the entire wavelength range according to the invention between 380 and 425 nm can also be used.

Furthermore, it has proven particularly advantageous if the wavelength of the pulsed treatment radiation is at 405 nm. Therein, the pulse duration of the individual laser pulses can again be in a range between 0.1 ps and 10 ns, in particular between 0.1 ps and 250 ps, at a pulse energy between 0.1 nJ and 5 μJ. The pulse duration range according to the invention from 0.1 ps to 250 ps means that the concerned pulse duration can be adjusted to the following values: 0.1 ps, 0.2 ps, 0.3 ps, 0.4 ps, 0.5 ps, 0.6 ps, 0.7 ps, 0.8 ps, 0.9 ps, 1.0 ps, 2 ps, 3 ps, 4 ps, 5 ps, 6 ps, 7 ps, 8 ps, 9 ps, 10 ps, 20 ps, 25 ps, 30 ps, 35 ps, 40 ps, 45 ps, 50 ps, 55 ps, 60 ps, 65 ps, 70 ps, 75 ps, 80 ps, 85 ps, 90 ps, 95 ps, 100 ps, 105 ps, 110 ps, 115 ps, 120 ps, 125 ps, 130 ps, 135 ps, 140 ps, 145 ps, 150 ps, 155 ps, 160 ps, 165 ps, 170 ps, 175 ps, 180 ps, 185 ps, 190 ps, 195 ps, 200 ps, 205 ps, 210 ps, 215 ps, 220 ps, 225 ps, 230 ps, 235 ps, 240 ps, 245 ps, 250 ps. Intermediate values are also conceivable. Such a laser device set up according to the invention as well as the method according to the invention can in particular be used in the photodisruption of the eye lens or also in cataract treatments. Besides the wavelength of 405 nm, of course, the entire wavelength range according to the invention between 380 and 425 nm can be used. The apparatus according to the invention and the method according to the invention have proven particularly advantageous if the laser device is arranged to use a wavelength of the treatment radiation of approximately 405 nm at a pulse duration of 0.1 ps to 10 ps and a pulse energy of the treatment radiation of 0.1 nJ to 5 μJ. Such an adjustment is in particular advantageous in the presbyopia treatment as well as again in the photodisruption of the eye lens. Here too, besides the wavelength of 405 nm, of course, the entire range according to the invention between 380 and 425 nm of the treatment radiation can be used.

The wavelength range according to the invention between about 380 nm and 425 nm means that the following wavelengths can be provided: 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm. By about 380 nm, wavelengths in a range between 375 nm and 380 nm are also to be understood. Furthermore, it has proven advantageous, as already partially described above, that the pulse duration of the treatment radiation is between 0.1 ps and 250 ps, between 1 ps and 250 ps, between 0.1 ps and 10 ps or between 0.1 ns and 10 ns. According to wavelength, pulse energy and also the type of the laser as well as the type of the surgical treatment of the eye to be performed, in particular the eye cornea, the mentioned pulse duration ranges can be applied. The same applies to the pulse repetition rate of the treatment radiation, which is at least 10 kHz, preferably between 100 kHz and 1 MHz, according to the invention.

In further advantageous configurations of the apparatus according to the invention, the laser device is a solid-state laser or a microchip laser. It is crucial that the used laser types are capable of emitting pulsed treatment radiation in the wavelength range between 380 and 425 nm. An indium gallium nitride laser is exemplarily mentioned for this. Of course, the emission of pulsed treatment radiation in the wavelength range between about 380 nm and 425 nm by a laser source with a higher fundamental wavelength can also be generated by frequency doubling, tripling, quadrupling etc. Since these are basically known methods and apparatuses, this is not to be elaborated in more detail at this point.

In further advantageous configurations of the apparatus according to the invention, it has at least one focusing device for focusing the treatment radiation on or in the eye cornea and/or at least one deflecting device for deflecting the treatment radiation from a laser source to the eye cornea. It is possible to exactly position the treatment radiation by the focusing device.

Control and monitoring devices for controlling and operating the laser device are known. In the method according to the invention, a surgical treatment can include generating flaps, rings, recesses or pockets in the eye cornea by means of the treatment radiation. Furthermore, the surgical treatment can include keratoplasty, photodisruption of the eye lens, cataract treatment, glaucoma treatment, a “cross-linking” method of the cornea or a presbyopia treatment.

Further features of the invention are apparent from the claims, the embodiment as well as based on the drawing. The features and feature combinations mentioned above in the description as well as the features and feature combinations mentioned below in the embodiments are usable not only in the respectively specified combination, but also in other combinations without departing from the scope of the invention.

### BRIEF DESCRIPTION OF THE DRAWING

The FIGURE shows a schematic diagram of an apparatus for performing surgical treatments of an eye, in particular an eye cornea.

### DETAILED DESCRIPTION OF EMBODIMENTS OF THE INVENTION

The FIGURE shows a schematic diagram of an apparatus 10 for performing a surgical treatment of an eye, in particular of an eye cornea 18. Therein, the apparatus 10 includes a laser device 12, which is arranged to emit pulsed treatment radiation 20 having a wavelength between about 380 nm and 425 nm and a pulse duration between 0.1 ps and 10 ns, wherein the pulse energy of the treatment radiation 20 is between 0.1 nJ and 5 μJ. One recognizes that the treatment radiation 20 emitted by the laser device 12 is directed from the laser device 12 serving as a laser source to the eye cornea 18 via a deflecting device 14. After the deflecting device 14, a focusing device 16 for focusing the treatment radiation 20 on or in the eye cornea 18 is disposed in the optical path. The thus controlled and focused pulses of the treatment radiation can then for example generate a flap or pocket in the eye cornea 18. Due to the relatively low pulse energies of 0.1 nJ to 5 μJ, excellent treatment results can be achieved within the scope of the surgical eye treatment in cooperation with the pulse duration of 0.1 ps to 10 ns, in particular 0.1 ps to 250 ps or 0.6 ps to 250 ps, and the wavelength range between 380 nm and 425 nm of the pulsed treatment radiation, in particular 380 nm and 405 nm.

We claim:

1. Apparatus for performing surgical treatments of an eye, in particular the eye cornea, including a laser device, which is arranged to emit pulsed treatment radiation having a wavelength between about 380 nm and 425 nm and a pulse duration between 0.1 ps and 10 ns, wherein the pulse energy of the treatment radiation is between 0.1 nJ and 5 μJ.

2. Apparatus according to claim 1, wherein the wavelength of the treatment radiation is between 380 and 405 nm.

3. Apparatus according to claim 1, wherein the wavelength of the treatment radiation is at 380 nm, 383 nm or 405 nm.

4. Apparatus according to claim 1, wherein the pulse duration is 0.1 ps to 250 ps, 1 ps to 250 ps, 0.1 ps to 10 ps or 0.1 ns to 10 ns.

5. Apparatus according to claim 1, wherein the pulse duration is 0.6 ps to 250 ps.

6. Apparatus according to claim 1, wherein the pulse repetition rate of the treatment radiation is at least 10 kHz.

7. Apparatus according to claim 1, wherein the pulse repetition rate of the treatment radiation is between 100 kHz and 1 MHz

8. Apparatus according to claim 1, wherein the laser device is a solid-state laser or a microchip laser.

9. Apparatus according to claim 1, wherein the apparatus has at least one focusing device for focusing the treatment radiation on or in the eye cornea.

10. Apparatus according to claim 1, wherein the apparatus includes at least one deflecting device for deflecting the treatment radiation from a laser source to the eye cornea.

11. Method for surgical treatment of an eye, in particular of the eye cornea, including the following method steps:
c) providing a laser device, which is arranged to emit pulsed treatment radiation having a wavelength between about 380 nm and 425 nm and a pulse duration between 0.1 ps and 10 ns, wherein the pulse energy of the treatment radiation is between 0.1 nJ and 5 μJ;
d) ablating predefined areas of the eye, in particular of the eye cornea, by means of the treatment radiation.

12. Method according to claim 11, wherein the wavelength of the treatment radiation is between 380 and 405 nm.

13. Method according to claim 11, wherein the wavelength of the treatment radiation is at 380 nm, 383 nm or 405 nm.

14. Method according to claim 11, wherein the pulse duration is 0.1 ps to 250 ps, 1 ps to 250 ps, 0.1 ps to 10 ps or 0.1 ns to 10 ns.

15. Method according to claim 11, wherein the pulse duration is 0.6 ps to 250 ps.

16. Method according to claim 11, wherein the pulse repetition rate of the treatment radiation is at least 10 kHz, preferably between 100 kHz and 1 MHz

17. Method according to claim 11, wherein the surgical treatment includes generating flaps, rings, recesses or pockets in the eye cornea by means of the treatment radiation.

18. Method according to claim 11, wherein the surgical treatment includes keratoplasty, photodisruption of the eye lens, cataract treatments, glaucoma treatment, a “cross-linking” method of the cornea or a presbyopia treatment.
